(19) United States
(12) Patent Application Publication
(10) Pub. No.: US 2026/0256622 A1
(43) Pub. Date: Sep. 3, 2026

(54) Eye Cover for Lid Closure

(71) Applicant: Ophthalmic Resources Partners, L.L.C, Sparks, NV (US)

(72) Inventors: Douglas K. Devries, Sparks, NV (US); Paul M. Karpecki, Lexington, KY (US)

(21) Appl. No.: 19/648,855

(22) Filed: Apr. 15, 2026

Related U.S. Application Data
(62) Division of application No. 17/581,209, filed on Jan. 21, 2022.
(60) Provisional application No. 63/254,711, filed on Oct. 12, 2021.
(60) Provisional application No. 63/230,534, filed on Aug. 6, 2021.

(51) Int. Cl. A61F 9/04; A61M 21/02

(52) U.S. Cl. CPC A61F 9/04; A61M 21/02; A61M 2210/0612

(57) ABSTRACT

The invention is directed to an eye cover for maintaining eye closure during sleep or for daytime wear for certain eye conditions. The eye cover is sized and shaped so as to fit within the orbit of the patient’s eye rather than around the orbit. By fitting closely within the orbit of the eye, the eye cover is much less likely to be disturbed or dislodged during movement over the course of a night’s sleep. The eye cover further is porous and includes fenestrations to provide for the passage of air to the eye.

### CROSS-REFERENCE TO RELATED APPLICATIONS

This application is a divisional of US Patent Application No. 17/581,209, filed on January 21, 2022, which claimed the benefit of US Provisional Patent Application Nos. 63/230,534, filed August 6, 2021, and 63/254,711, filed October 12, 2021. Each of these applications are incorporated by reference as if fully set forth herein.

### BACKGROUND

Studies have shown that forty to seventy percent of persons over the age of forty-five who suffer from dry eye disease have nighttime eyelid closure issues that cause nighttime exposure of the eyes. The lack of eyelid closure could lead to exposure keratitis and desiccation of the ocular surface. It also may contribute to meibomian gland dysfunction, which is the leading cause of dry eye disease. Inadequate eyelid closure at night is also a significant contributor to dry eye disease and is present in the vast majority of patients with non-responsive dry eye disease as well as those with morning symptoms. Such exposure may also be due to a variety of incomplete eyelid closure issues such as nocturnal lagophthalmos, eyelid misalignment, or eyelid paralytic conditions. Many of the above conditions are directly associated with or a direct cause of dry eye disease.

Patches or eye covers are known for various other purposes, including for placement over eyes during surgery while the patient is under anesthesia. One such eye cover is taught by US Patent No. 5,887,590. These eye covers fit over the entire eye and attach to the skin by adhesive around the outside of the orbit of the eye. Since the patient does not move while under anesthesia, these surgical eye covers are not designed to stay in place in response to movement of the patient’s head. For this reason, such eye covers are not well adapted for use during sleep. Eye covers of this design may be displaced as the patient turns or moves during the course of a night’s sleep, potentially causing discomfort or even injury to the patient.

Ordinary tape has also been used to hold eyes closed during sleep, but this may present a problem because eyelashes may stick to the adhesive of the tape. In addition, patients may develop skin sensitivities to the adhesive over time. Furthermore, removal of the tape each morning may be uncomfortable for the patient, or displace eye lashes, thereby discouraging use.

A better solution for preventing exacerbation of dry eye disease from exposure during sleep or for certain conditions during daytime is therefore desired.

### SUMMARY

The invention is directed to an eye cover for maintaining complete and essential eye closure during sleep. The eye cover is sized and shaped so as to fit within the orbit of the patient’s eye rather than around the orbit. By fitting closely within the orbit of the eye, the eye cover is much less likely to be disturbed or dislodged during movement over the course of a night’s sleep. The eye cover further is porous by means of fenestrations to provide for the passage of air to minimize overactivation of the sebaceous and sudoriferous glands surrounding the skin of the eye and face.

In addition to nighttime eye closure, the invention in certain embodiments is efficacious in patients requiring lid closure during the day, such as Bell’s palsy patients, after the use of cryopreserved or dehydrated amniotic membrane, for patients requiring any type of temporary tarsorrhaphy where lids must remain closed throughout the day, and patients requiring a pressure patch bandage during the day and/or night.

It is therefore an object of the invention to provide an eye cover for use by persons suffering from conditions such as dry eye disease in order to maintain eye closure during sleep or during the day to treat certain conditions.

It is a further object of the invention to provide an eye cover that fits within the orbit of the eye and therefore is not easily dislodged as a result of movement such as during sleep.

It is a further object of the invention to provide an eye cover that allows ample passage of air through the cover while maintaining complete eye closure.

It is a further object of the invention to provide an eye cover that can be easily removed after use without discomfort or lash loss to the patient.

These and other features, objects and advantages of the present invention will become better understood from a consideration of the following detailed description of the preferred embodiments and appended claims in conjunction with the drawings as described following:

### DRAWINGS

FIG. 1 is a plan view of an eye cover according to an embodiment of the present invention.

FIG. 2 is a plan view of a pair of eye covers on a paper backing according to an embodiment of the present invention

FIG. 3 is a perspective view of an eye cover fitted to a patient according to an embodiment of the present invention.

FIG. 4 is a perspective view of an eye cover being applied by a patient according to an embodiment of the present invention.

### DETAILED DESCRIPTION OF THE PREFERRED EMBODIMENTS

Before the present invention is described in further detail, it should be understood that the invention is not limited to the particular embodiments described, and that the terms used in describing the particular embodiments are for the purpose of describing those particular embodiments only, and are not intended to be limiting, since the scope of the present invention will be limited only by the claims.

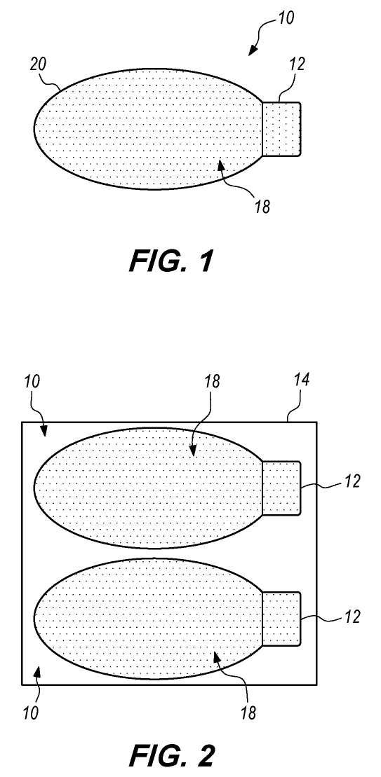

As shown in FIG. 1, an eye cover 10 according to an embodiment of the invention is designed to safely reduce or eliminate exposure of the corneal and conjunctival tissue of the eye during the sleeping hours or during the day to treat certain conditions. The eye cover 10 in an embodiment of the invention is a latex free, hypoallergenic means of keeping the lid closed for extended periods of time by having a shape that allows for minimal contact with the patient’s skin. In particular, main section 20 of eye cover 10 comprises an asymmetric contour shape to conform to the orbit of the eye adnexa of patient 10 with a narrow medial canthus and a wider temporal canthus. The eye cover 10 also features a small tab 12 for removal, which extends laterally from main section 20 of eye cover 10. Main section 20 of eye cover 10 may be formed of a flexible plastic material, including in certain embodiments polypropylene, polyethylene, an acrylic polymer, and polyvinyl alcohol.

The latex free, hypoallergenic flexible material for main section 20 of eye cover 10 is a porous plastic and also includes fenestrations 18. The porosity and fenestrations 18 allow the least possible surface area of main section 20 of eye cover 10 being in contact with the skin of patient 16 while maintaining lid closure. The porous material and the fenestrations 18 combined with the minimal coverage of contour shaping allows the skin of patient 16 to breathe and reduces the activation of the sudoriferous and sebaceous glands. This prevents decreases in adhesion of main section 20 of eye cover 10 by minimizing the secretion of oil and moisture from the glands, thus enabling the device to be worn for extended periods of time such as through a full night of sleep. The contoured shape of main section 20 of eye cover 10 improves wearability by reducing irritation by not having redundant material while being worn for the extended periods of time required to prevent exposure of the corneal and conjunctival tissue.

In an alternative embodiment, a sensitive version of the eye cover 10 can have all of the features of the standard eye cover 10 according to an embodiment as described herein but is made with less adhesive on main section 20 of eye cover 10 for patients 16 with ultra-sensitive skin that have demonstrated reactions to adhesives in the past or the standard version is too adherent on the skin tissue or lashes. In such embodiments, adhesive may not cover the entire lower side of main section 20 of eye cover 10. The adhesive may, for example, extend around only the periphery of the lower side of main section 20 of eye cover 10.

In alternative versions of the two embodiments just described, either or both made be made with the original porous plastic either with or without the added fenestrations. The versions without fenestrations may be used for patients who are suitable and require additional assistance in moisture retention.

In a particular embodiment, main section 20 of eye cover 10 may be 28mm wide, which provides a lower total surface area and allows better adhesion within the orbit area of patient 16 and less opportunity for movement during sleep to dislodge an edge of main section 20 of eye cover 10. This makes eye cover 10 more suitable for overnight wear, as needed with chronic lid closure issues. Main section 20 of eye cover 10 still, however, is of sufficient length to cover the eye of patient 16, and thus at least the length of the eye of patient 16, and should be of sufficient width to cover the upper and lower eyelid of patient 16. In a particular embodiment, eye cover 10 may extend for 64mm in length, including the tab 12 at one corner which may be 10mm in length while main section 20 of eye cover 10 is 54mm in length. The precise length of main section 20 of eye cover 10 may be varied in a size range from 44-56mm. The precise width of eye cover 10 may be varied in a size range of 22-32mm in order to be adapted to patients 16 with larger or smaller orbit structures. Other dimensions may be likewise varied. For example, some persons of Asian descent may be more likely to find that a smaller size of eye cover 10 fits more comfortably within the orbit of the eye.

The adhesive used on the lower side of main section 20 of eye cover 10 according to certain embodiments is a pressure-sensitive water-soluble gelatinous or hydrocolloidal adhesive which can be easily removed without sticking to the eyelashes of patient 16 and can be used with comfort. A natural or synthetic hydrocolloid provides sufficient stickiness to cause the eye cover 10 to adhere sufficiently during sleep. Suitable synthetic and natural water-soluble hydrocolloids and gelatins include karaya gum, guar gum, collagen, polysaccharide gum, locust bean gum, powdered pectin, gelatin, carboxymethyl cellulose and the like.

Fenestrations 18 in eye cover 10 may be, in certain embodiments, distributed evenly across the surface of eye cover 10 in a regular pattern, or distributed evenly across only the surface of main section 20 of eye cover 10. For example, the fenestrations 18 may be situated 1mm apart from each other in a regular grid pattern across the surface of eye cover 10 and passing entirely through eye cover 10. Using a particular size of eye cover 10 as described above, this would yield in excess of 800 fenestrations 18 across such embodiment of eye cover 10. The shape, size, number, and arrangement of fenestrations 18 may be varied in alternative embodiments.

Eye cover 10 may be distributed for use by patient 16 using a paper backing 14, as shown in FIG. 2. For example, two eye covers 10 may be adhered to each paper backing 14 for ease of use. The paper backing 14 is formulated with a non-stick surface such that the adhesive on the lower side of main section 20 of eye cover 10 still allows for easy removal of each eye cover 10 from paper backing 14 by the patient without any part of paper backing 14 remaining on eye cover 10 after it is removed. Tab 12 has no adhesive on its lower side, and thus tab 12 may be used by patient 16 to easily remove eye cover 10 from paper backing 14. In alternative embodiments, paper backing 16 may be formed of a material other than paper, such as with cardstock or various plastics.

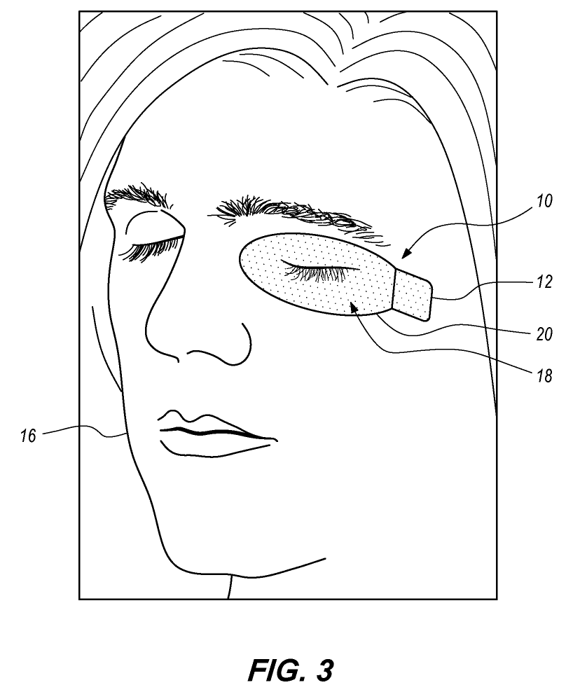

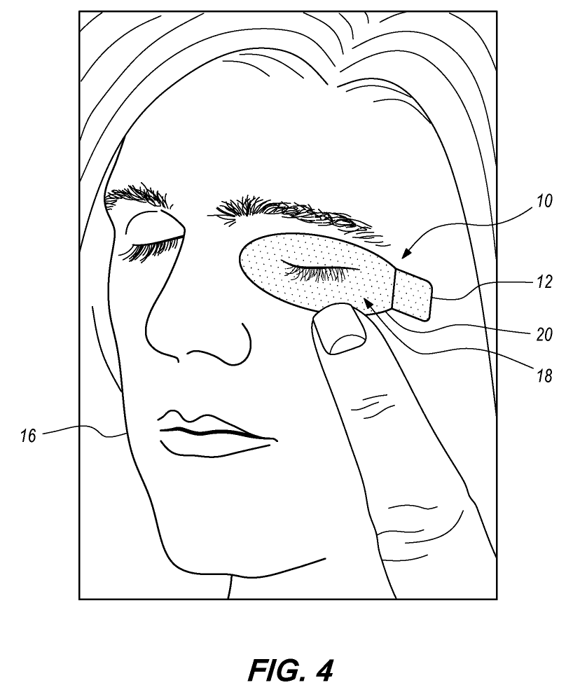

Use of the eye cover is illustrated in FIGS. 3 and 4. Patient 16 should first apply any medication to the eye as prescribed by a physician and excess medication wiped away. The patient 16 then removes eye cover 10 from paper backing 16 by gripping adhesive-free tab 12, being careful not to touch the surfaces of the eye cover 10 before applying to the eye of patient 16. Main section 20 of eye cover 10 is then placed over the closed eye of patient 16. The tab 12 is oriented laterally. The patient 16 then gently seals eye cover 10 by running a finger around the entire edge of main section 20 of eye cover 10. The process is repeated for the second eye, if patient 16 is applying an eye cover 10 to both eyes.

Eye cover 10 may be removed by gently grasping tab 12 and pulling in a sideward direction so that the eye of patient 16 is not opened during removal of eye cover 10. In an embodiment, eye cover 10 is single use only, and should therefore be discarded after use.

As will be apparent to those skilled in the art, and as indicated above, many modifications and variations of the foregoing detailed description are possible within the spirit and scope of the present invention.

Unless otherwise stated, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein. It will be apparent to those skilled in the art that many more modifications are possible without departing from the inventive concepts herein.

All terms used herein should be interpreted in the broadest possible manner consistent with the context. When a grouping is used herein, all individual members of the group and all combinations and subcombinations possible of the group are intended to be individually included. When a range is stated herein, the range is intended to include all subranges and individual points within the range. All references cited herein are hereby incorporated by reference to the extent that there is no inconsistency with the disclosure of this specification.

The present invention has been described with reference to certain preferred and alternative embodiments that are intended to be exemplary only and not limiting to the full scope of the present invention, as set forth in the appended claims.

1. A method of maintaining eyelid closure in a patient, comprising:
removing an eyelid seal comprising a main section and a tab from a backing by gripping the tab;
placing the main section over a closed eye of the patient with the tab oriented laterally, wherein the main section is applied wholly within the orbit of the closed eye, wherein the main section comprises a width sufficient to cover an upper and lower eyelid of the patient and a length at least equal to a length of the eye of the patient, but wherein the main section width and main section length are limited such that the main section fits wholly within an orbit of the eye of the patient, further wherein the main section comprises an asymmetric contour shape to conform to the orbit of the eye of the patient with a medial canthus of the eye narrower than a temporal canthus of the eye, further wherein a lower side of the main section comprises an adhesive of sufficient stickiness to maintain eyelid closure for the patient during a night’s sleep, further wherein the tab comprises a tab lower side free of the adhesive;
sealing the eyelid seal by running a finger around an entire edge of the main section;
maintaining the eyelid seal in place during a period of sleep of the patient to prevent nighttime exposure of the eye due to an improper lid closure; and
removing the eyelid seal after the period of sleep by grasping the tab and pulling in a sideward direction.

2. The method of claim 1, wherein the eyelid seal further comprises a plurality of fenestrations that allow passage of air through the eyelid seal while maintaining complete lid seal.

3. The method of claim 2, wherein the main section of the eyelid seal comprises a porous plastic material that allows the skin of the patient to breathe and reduces activation of sudoriferous and sebaceous glands during sleep.

4. The method of claim 2, wherein the adhesive is a water-soluble gelatinous or hydrocolloidal adhesive that prevents eyelashes from sticking to the eyelid seal during sleep.

5. The method of claim 4, further comprising applying the eyelid seal to a patient having a nighttime eyelid closure issue selected from the group consisting of nocturnal lagophthalmos, eyelid misalignment, and eyelid paralytic conditions.

6. The method of claim 1, further comprising:
applying medication to the eye prior to applying the eyelid seal; and
wiping away excess medication before applying the eyelid seal.

7. The method of claim 6, wherein maintaining the eyelid seal in place prevents or reduces meibomian gland dysfunction.
